(19) United States
(12) Patent Application Publication
(10) Pub. No.: US 2014/0296263 A1
(43) Pub. Date: Oct. 2, 2014

(54) SUBSTITUTED XANTHINE DERIVATIVES

(71) Applicants: Ara Aslanian, Arlington, MA (US); Dolly Parasrampuria, Marlborough, MA (US); Kristine Hogan, Danvers, MA (US); Virginia Braman, Lexington, MA (US); Changfu Cheng, Northborough, MA (US)

(72) Inventors: Ara Aslanian, Arlington, MA (US); Dolly Parasrampuria, Marlborough, MA (US); Kristine Hogan, Danvers, MA (US); Virginia Braman, Lexington, MA (US); Changfu Cheng, Northborough, MA (US)

(73) Assignee: CONCERT PHARMACEUTICALS, INC., Lexington, MA (US)

(21) Appl. No.: 14/233,590

(22) PCT Filed: Jul. 19, 2012
(86) PCT No.: PCT/US2012/047418

§ 371 Date: Jun. 4, 2014

Related U.S. Application Data
(60) Provisional application No. 61/509,343, filed on Jul. 19, 2011.
(60) Provisional application No. 61/607,286, filed on Mar. 6, 2012.

(51) Int. Cl. C07D 473/10

(52) U.S. Cl. CPC C07D 473/10; C07B 2200/05
USPC 514/263.36; 544/267

(57) ABSTRACT

The present invention in one embodiment relates to a compound of Formula (I); or a pharmaceutically acceptable salt thereof, wherein: each of R¹ and R² is independently selected from —CH₃ and —CD₃; R³ is hydrogen or deuterium; each of Z¹, Z² and Z³ is independently selected from hydrogen and deuterium; each Z⁴ is hydrogen or deuterium; each Z⁵ is hydrogen or deuterium; and each Z⁶ is hydrogen or deuterium.

### RELATED APPLICATIONS

The present application claims benefit of priority to U.S. Provisional Application No. 61/509,343, filed on Jul. 19, 2012; and U.S. Provisional Application No. 61/607,286, filed on Mar. 6, 2012.

### BACKGROUND OF THE INVENTION

Pentoxifylline, 1-(5-oxohexyl)-3,7-dimethylxanthine, is sold under the name Trental® in the U.S. and Canada. It is currently approved for the treatment of patients with intermittent claudication on the basis of chronic occlusive arterial disease of the limbs. It is also in clinical trials for glomerulonephritis, nephrotic syndrome, nonalcoholic steatohepatitis, Leishmaniasis, cirrhosis, liver failure, Duchenne's muscular dystrophy, HIV infection, late radiation induced injuries, radiation induced lymphedema, alcoholic hepatitis, radiation fibrosis, necrotizing enterocolitis in premature neonates, chronic kidney disease, pulmonary sarcoidosis, recurrent aphthous stomatitis, chronic breast pain in breast cancer patients, brain and central nervous system tumors, and malnutrition-inflammation-cachexia syndrome. Pentoxifylline has also recently garnered attention as a potential treatment for diabetes and disorders associated with diabetes. See Ferrari, E et al., Pharmatherapeutica, 1987, 5(1): 26-39; Raptis, S et al., Acta Diabetol Lat, 1987, 24(3):181-92; and Rahbar, R et al., Clin Chim Acta, 2000, 301(1-2): 65-77.

Pentoxifylline is known to have activity as an inhibitor of phosphodiesterase (PDE; see Meskini, N et al., Biochem. Pharm. 1994, 47(5): 781-788) as well as activity against other biological targets, but its exact mode of action leading to clinical effects is unknown. Pentoxifylline has been shown to improve blood flow properties through hemorheologic effects which lower blood viscosity and improve erythrocyte flexibility. Pentoxifylline also increases leukocyte deformability and inhibits neutrophil adhesion and activation. (See FDA label for pentoxifylline at http://www.fda.gov/cder/foi/nda/99/74-962_Pentoxifylline_prntlbl.pdf). In addition to improving hemorheologic properties, pentoxifylline is also believed to have anti-inflammatory and anti-fibrotic properties.

The clinical pharmacology of pentoxifylline has been attributed to the parent drug as well as its metabolites, notably the M-1 metabolite, though the sequence of events leading to clinical improvement is still to be defined. Pentoxifylline undergoes rapid first pass metabolism. Peak plasma levels of pentoxifylline and its metabolites are reached within one hour. Structures of pentoxifylline (shown as Compound 400 below) and its various reported metabolites are shown below.

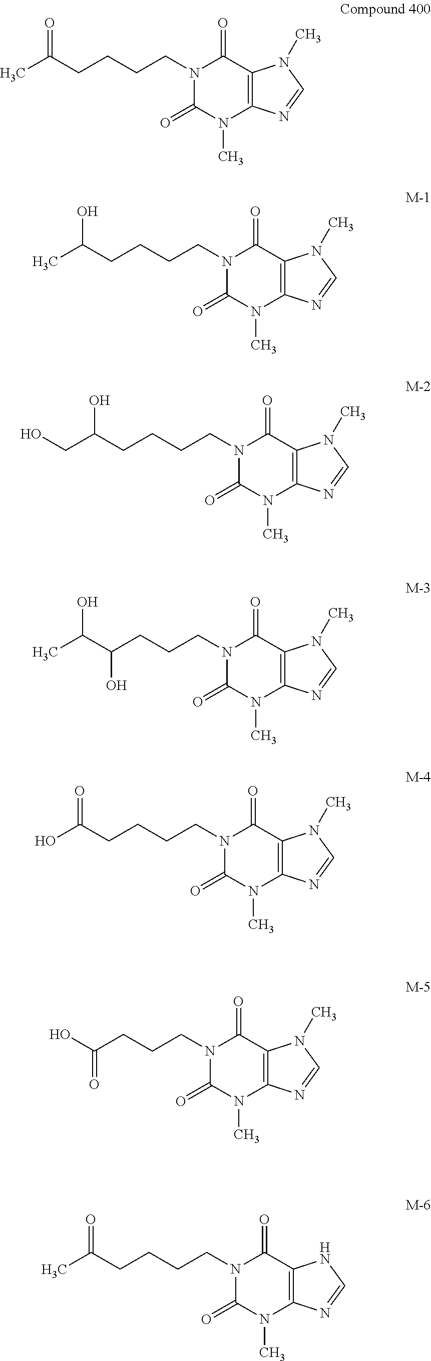

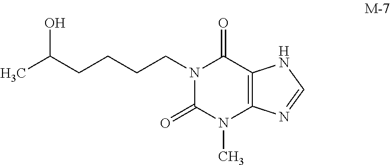 [SMILES: CC(O)CCCCn1c(=O)c2[nH]cnc2n(C)c1=O]

U.S. patent application Ser. No. 12/380,579 describes deuterium-substituted analogs of pentoxifylline and its active M-1 metabolite. For certain of these analogs, the deuterium substitution is reported to provide enhanced metabolic stability as well as a lower amount of the unwanted M-5 metabolite.

Recently, a deuterated version of the M-1 metabolite known as CTP-499 was advanced into Phase 1 clinical studies for the treatment of diabetic nephropathy and more generally chronic kidney disease. See www.concertpharma.com/CTP499Phase1.htm. From an assessment of the pharmacokinetic behavior of CTP-499 in healthy human volunteers it has now been found that following administration of CTP-499, there is a substantial increase in plasma levels of the M-2 metabolite relative to its plasma levels following administration of pentoxifylline. The biological activity of the M-2 metabolite itself was not previously known. Based on the pharmacokinetic studies with CTP-499 in humans showing higher levels of M-2, the metabolite was prepared and subsequently evaluated. It was found to have in vitro activity against inflammatory and fibrotic targets such as inhibition of Lipopolysaccharide (LPS)-induced production of tumor necrosis factor-alpha (TNF-α) activity in whole blood. These studies suggest that the M-2 metabolite may contribute to the overall pharmacology of CTP-499 and, to a lesser extent pentoxifylline, and it may play a role in the ability of CTP-499 to treat diabetic nephropathy and more generally chronic kidney disease.

Kidney disease is growing health concern. According to the National Kidney Foundation, 26 million Americans suffer from chronic kidney disease and millions of others are at increased risk. Kidney disease progresses through stages. At end stage kidney failure, when 85-90 percent of kidney function is lost, dialysis is needed. The number of patients afflicted with end stage renal disease has grown rapidly in recent years; over the ten year period ending in 2006 the number increased by 64%.

Despite the available treatments for chronic kidney disease, there is a continuing need for new agents that are safe and effective, especially agents that have the potential to treat the disease with different mechanisms of action.

### SUMMARY OF THE INVENTION

This invention relates to novel compounds that are substituted xanthine derivatives and pharmaceutically acceptable salts thereof. In particular, this invention relates to a metabolite of pentoxifylline, 1-(5,6-dihydroxyhexyl)-3,7-dimethyl-xanthine, and deuterium-substituted analogs thereof. This invention also provides compositions comprising one or more compounds of this invention and a carrier and the use of the disclosed compounds and compositions in methods of treating inflammatory and fibrotic diseases such as chronic kidney disease. The invention also relates to a method of delivering the metabolite to a patient in need thereof by administering a therapeutic agent that forms the metabolite in the body.

### BRIEF DESCRIPTION OF THE DRAWINGS

FIG. 1 shows the relative potency (calculated as disclosed herein) of Compounds 401, 400 (pentoxifylline), M2 (the M2 metabolite of pentoxifylline, also indicated as compound 308 in this application) M3, M4 and M5 in TNF-α inhibition and reactive oxygen species (ROS) inhibition assays. For each compound, the relative potency in the TNF-α inhibition assay is shown as the left-hand-side bar, while the relative potency in the ROS inhibition assay is shown as the right-hand-side bar.

### DETAILED DESCRIPTION OF THE INVENTION

### Definitions

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention unless otherwise specified any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The pharmaceutically acceptable salt may also be a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers, even more preferably less than 2% of other stereoisomers, even more preferably less than 1% of other stereoisomers, even more preferably less than 0.5% of other stereoisomers, even more preferably less than 0.1% of other stereoisomers, even more preferably less than 0.05% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “mammal” as used herein includes a human or a non-human animal. In one embodiment, the mammal is a non-human animal. In another embodiment, the mammal is a human.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

“Substituted with deuterium” refers to the replacement of one or more hydrogen atoms with a corresponding number of deuterium atoms.

Throughout this specification, a variable may be referred to generally (e.g., “each Z”) or may be referred to specifically (e.g., Z¹, Z², Z³, etc.). Unless otherwise indicated, when a variable is referred to generally, it is meant to include all specific embodiments of that particular variable (for example, “Z¹” includes both Z^{1a} and Z^{1b}).

### Therapeutic Compounds

The present invention in one embodiment relates to a compound of Formula I:

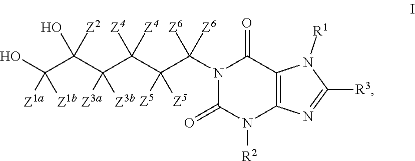

or a pharmaceutically acceptable salt thereof, wherein:

each of R¹ and R² is independently selected from —CH₃ and —CD₃;

R³ is hydrogen or deuterium;

each of Z¹, Z² and Z³ is independently selected from hydrogen and deuterium;

each Z⁴ is hydrogen or deuterium;

each Z⁵ is hydrogen or deuterium; and

each Z⁶ is hydrogen or deuterium.

One embodiment relates to a compound of formula I where the variables R¹, R², R³, Z¹, Z², Z³, Z⁴, Z⁵ and Z⁶ are as described above, provided that at least one of R¹ and R² is —CD₃ or at least one of R³, Z¹, Z², Z³, Z⁴, Z⁵ and Z⁶ is deuterium.

Another embodiment relates to a compound of formula I wherein each Z⁴, Z⁵ and Z⁶ is hydrogen. In one aspect of this embodiment, each of R¹ and R² is —CH₃ and R³ is hydrogen.

Another embodiment relates to a compound of formula I wherein each Z², Z⁴, Z⁵ and Z⁶ is hydrogen. In one aspect of this embodiment, each of R¹ and R² is —CH₃ and R³ is hydrogen.

Another embodiment relates to a compound of formula I wherein each Z¹ and Z³ is deuterium and each Z², Z⁴, Z⁵ and Z⁶ is hydrogen. In one aspect of this embodiment, each of R¹ and R² is —CH₃ and R³ is hydrogen.

Another embodiment relates to a pharmaceutical composition comprising a compound of formula I or a pharmaceutically acceptable salt thereof and a pharmaceutically acceptable carrier. In further embodiments, the pharmaceutical composition comprises one of the aforementioned embodiments of a compound of formula I.

In formula I, the carbon bearing the Z² substituent is asymmetric. Thus, the present compounds may exist as a racemic mixture or as predominantly one enantiomer in either the (S) or (R) configuration at the carbon bearing the Z² substituent. When a compound of formula I exists as predominantly one enantiomer, the S enantiomer is preferred. Examples of specific compounds of formula I are shown in Table 1 below.

TABLE 1
Examples of Specific Compounds of Formula I

| | |
|---|---|
| 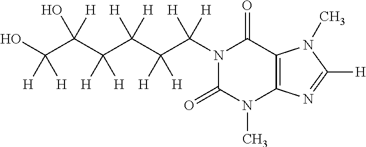 [SMILES: Cn1cnc2c1c(=O)n(CCCCC(O)CO)c(=O)n2C] | I-a |
| 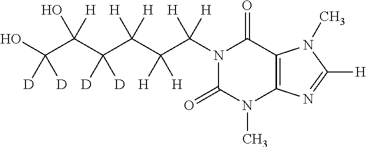 [SMILES: [2H]C([2H])(O)C(O)C([2H])([2H])CCCn1c(=O)c2c(ncn2C)n(C)c1=O] | I-b |
| 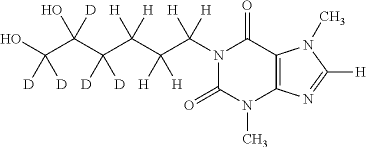 [SMILES: [2H]C([2H])(O)C([2H])(O)C([2H])([2H])CCCn1c(=O)c2c(ncn2C)n(C)c1=O] | I-c |
| 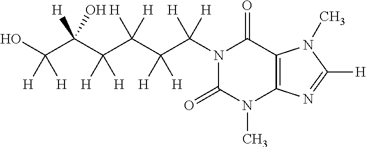 [SMILES: Cn1cnc2c1c(=O)n(CCCC[C@@H](O)CO)c(=O)n2C] | I-d |
| 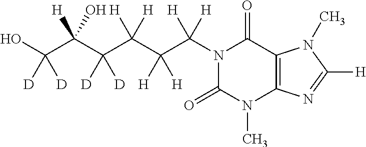 [SMILES: [2H]C([2H])(O)[C@H](O)C([2H])([2H])CCCn1c(=O)c2c(ncn2C)n(C)c1=O] | I-e |
| 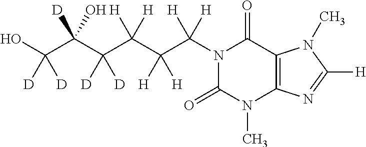 [SMILES: [2H]C([2H])(O)[C@]([2H])(O)C([2H])([2H])CCCn1c(=O)c2c(ncn2C)n(C)c1=O] | I-f |
| 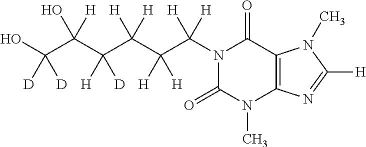 [SMILES: [2H]C(CCCn1c(=O)c2c(ncn2C)n(C)c1=O)C(O)C([2H])([2H])O] | I-g |
| 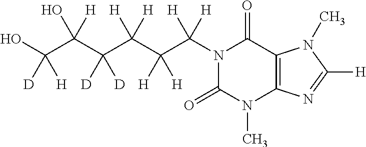 [SMILES: [2H]C(O)C(O)C([2H])([2H])CCCn1c(=O)c2c(ncn2C)n(C)c1=O] | I-h |
| 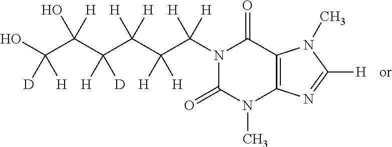 [SMILES: [2H]C(O)C(O)C([2H])CCCn1c(=O)c2c(ncn2C)n(C)c1=O ; "or"] | I-i |
| a pharmaceutically acceptable salt thereof. | |

Compound I-a is also the M-2 metabolite of compound 400 (pentoxifylline) and is also referred to as compound 308 in this application.

Examples of specific compounds of formula I also include the enantiomers of I-d, I-e and I-f or pharmaceutically acceptable salts thereof.

In another set of embodiments, any atom not designated as deuterium in any of the embodiments set forth above is present at its natural isotopic abundance.

In another embodiment, the compounds described in this section entitled “Therapeutic Compounds” are substantially pure and/or in isolated form, e.g., greater than 50%, 60%, 70%, 80%, 90%, 95%, 97%, 99%, 99.5% or 99.9% pure by weight. “Percent purity by weight” means the weight of the compound divided by the weight of the compound plus impurities times 100%. For example, in one aspect of this embodiment, a compound selected from the group consisting of compounds I-a, I-b, I-c, I-d, I-e, I-f, I-g, I-h and I-i, or a pharmaceutically acceptable salt thereof, is greater than 50%, 60%, 70%, 80%, 90%, 95%, 97%, 99%, 99.5% or 99.9% pure by weight.

As used herein, “isolated” means that the compounds described herein are separated from other components of either: (a) a natural source, such as a human or cell, preferably plasma, or (b) a synthetic organic chemical reaction mixture.

### Exemplary Synthesis

Compounds of Formula I, wherein Z², each Z⁴, Z⁵, and Z⁶ is hydrogen, may be prepared as outlined in Scheme 1 below utilizing appropriately deuterated starting materials as would be readily apparent to one of skill in the art.

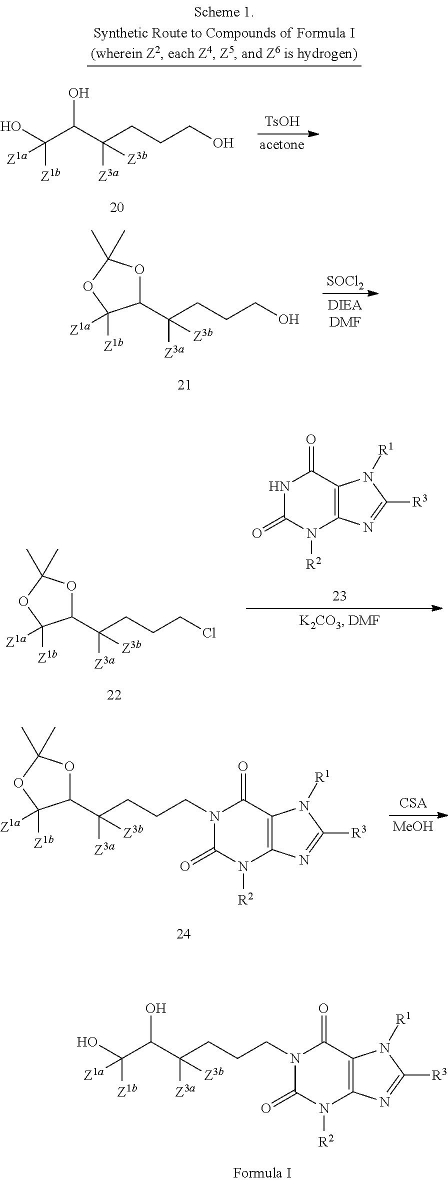

The synthesis of compound 308, the compound of formula I in which each Z is hydrogen, R³ is hydrogen, R¹ and R² are each CH₃, and any atom not designated as deuterium is at its natural isotopic abundance, is disclosed in Scheme 2.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of this invention and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pharmaceutical compositions comprising an effective amount of a compound of Formula I or a pharmaceutically acceptable salt thereof; and an acceptable carrier. In one embodiment, the pharmaceutical composition is pyrogen-free. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

In one embodiment of the compositions disclosed herein, the compound of Formula I is a compound disclosed in Table 1 above.

In another embodiment, a composition of this invention further comprises a second therapeutic agent. The second therapeutic agent may be selected from any compound or therapeutic agent known to have or that demonstrates advantageous properties when administered with a compound having the same mechanism of action as pentoxifylline. Such agents include those indicated as being useful in combination with pentoxifylline, including but not limited to, those described in WO 1997019686, EP 0640342, WO 2003013568, WO 2001032156, WO 2006035418, and WO 1996005838.

Preferably, the second therapeutic agent is an agent useful in the treatment or prevention of a disease or condition selected from peripheral obstructive vascular disease; glomerulonephritis; nephrotic syndrome; nonalcoholic steatohepatitis; Leishmaniasis; cirrhosis; liver failure; Duchenne's muscular dystrophy; late radiation induced injuries; radiation induced lymphedema; radiation-associated necrosis; alcoholic hepatitis; radiation-associated fibrosis; necrotizing enterocolitis in premature neonates; diabetic nephropathy, hypertension-induced renal failure, and other chronic kidney disease; Focal Segmental Glomerulosclerosis; pulmonary sarcoidosis; recurrent aphthous stomatitis; chronic breast pain in breast cancer patients; brain and central nervous system tumors; malnutrition-inflammation-cachexia syndrome; interleukin-1 mediated disease; graft versus host reaction and other allograft reactions; diet-induced fatty liver conditions, atheromatous lesions, fatty liver degeneration and other diet-induced high fat or alcohol-induced tissue-degenerative conditions; human immunodeficiency virus type 1 (HIV-1) and other human retroviral infections; multiple sclerosis; cancer; fibroproliferative diseases; fungal infection; drug-induced nephrotoxicity; collagenous colitis and other diseases and/or conditions characterized by elevated levels of platelet derived growth factor (PDGF) or other inflammatory cytokines; endometriosis; optic neuropathy and CNS impairments associated with acquired immunodeficiency syndrome (AIDS), immune disorder diseases, or multiple sclerosis; autoimmune disease; upper respiratory viral infection; depression; urinary incontinence; irritable bowel syndrome; septic shock; Alzheimers Dementia; neuropathic pain; dysuria; retinal or optic nerve damage; peptic ulcer; insulin-dependent diabetes; non-insulin-dependent diabetes; diabetic nephropathy; metabolic syndrome; obesity; insulin resistance; dyslipidemia; pathological glucose tolerance; hypertension; hyperlipidemia; hyperuricemia; gout; hypercoagulability; and inflammation or injury associated with neutrophil chemotaxis and/or degranulation. The compounds of this invention can also be used to control intraocular pressure or to stabilize auto-regulation of cerebral blood flow in subjects who require such control as determined by medical examination.

In one embodiment, the second therapeutic agent is selected from an angiotensin-converting enzyme (ACE) inhibitor and an angiotensin receptor blocker (ARB). Specific examples of ACE inhibitors include, but are not limited to, benazepril (lotensin), captopril (capoten), enalapril (vasotec), fosinopril (monopril), lisinopril (prinivil, zestril), moexipril (univasc), perindopril (aceon), quinapril (accupril), ramapril (altace), and trandolapril (mavik). Specific examples of ARBs include, but are not limited to, candesartan (atacand), eprosartan (teveten), irbesartan (avapro), losartan (cozaar), olmesartan (benicar), telmisartan (micardis) and valsartan (diovan).

In one embodiment, the second therapeutic agent is selected from α-tocopherol and hydroxyurea.

In another embodiment, the second therapeutic agent is useful in the treatment of diabetes or an associated disorder, and is selected from insulin or insulin analogues, glucagon-like-peptide-1 (GLP-1) receptor agonists, sulfonylurea agents, biguanide agents, alpha-glucosidase inhibitors, PPAR agonists, meglitinide agents, dipeptidyl-peptidase (DPP) IV inhibitors, other phosphodiesterase (PDE1, PDE5, PDE9, PDE10 or PDE1) inhibitors, amylin agonists, CoEnzyme A inhibitors, and antiobesity agents.

Specific examples of insulin include, but are not limited to Humulin® (human insulin, rDNA origin), Novolin® (human insulin, rDNA origin), Velosulin® BR (human buffered regular insulin, rDNA origin), Exubera® (human insulin, inhaled), and other forms of inhaled insulin, for instance, as delivered by Mannkind's “Technosphere Insulin System”.

Specific examples of insulin analogues include, but are not limited to, novarapid, insulin detemir, insulin lispro, insulin glargine, insulin zinc suspension and Lys-Pro insulin.

Specific examples of Glucagon-Like-Peptide-1 receptor agonists include, but are not limited to BIM-51077 (CAS-No. 275371-94-3), EXENATIDE (CAS-No. 141758-74-9), CJC-1131 (CAS-No. 532951-64-7), LIRAGLUTIDE (CAS-No. 20656-20-2) and ZP-10 (CAS-No. 320367-13-3).

Specific examples of sulfonylurea agents include, but are not limited to, TOLBUTAMIDE (CAS-No. 000064-77-7), TOLAZAMIDE (CAS-No. 001156-19-0), GLIPIZIDE (CAS-No. 029094-61-9), CARBUTAMIDE (CAS-No. 000339-43-5), GLISOXEPIDE (CAS-No. 025046-79-1), GLISENTIDE (CAS-No. 032797-92-5), GLIBORNURIDE (CAS-No. 026944-48-9), GLIBENCLAMIDE (CAS-NO. 010238-21-8), GLIQUIDONE (CAS-No. 033342-05-1), GLIMEPIRIDE (CAS-No. 093479-97-1) and GLICLAZIDE (CAS-No. 021187-98-4).

A specific example of a biguanide agent includes, but is not limited to METFORMIN(CAS-No. 000657-24-9).

Specific examples of alpha-glucosidase-inhibitors include, but are not limited to ACARBOSE (Cas-No. 056180-94-0), MIGLITOL (CAS-No. 072432-03-2) and VOGLIBOSE (CAS-No. 083480-29-9).

Specific examples of PPAR-agonists include, but are not limited to MURAGLITAZAR(CAS-No. 331741-94-7), ROSIGLITAZONE (CAS-NO. 122320-73-4), PIOGLITAZONE (CAS-No. 111025-46-8), RAGAGLITAZAR(CAS-NO. 222834-30-2), FARGLITAZAR(CAS-No. 196808-45-4), TESAGLITAZAR(CAS-No. 251565-85-2), NAVEGLITAZAR(CAS-No. 476436-68-7), NETOGLITAZONE (CAS-NO. 161600-01-7), RIVOGLITAZONE (CAS-NO. 185428-18-6), K-1 11 (CAS-No. 221564-97-2), GW-677954 (CAS-No. 622402-24-8), FK-614 (CAS-No 193012-35-0) and (−)-Halofenate (CAS-No. 024136-23-0). Preferred PPAR- agonists are ROSGLITAZONE and PIOGLITAZONE.

Specific examples of meglitinide agents include, but are not limited to REPAGLINIDE (CAS-No. 135062-02-1), NATEGLINIDE (CAS-No. 105816-04-4) and MITIGLINIDE (CAS-No. 145375-43-5).

Specific examples of DPP IV inhibitors include, but are not limited to SITAGLIPTIN(CAS-No. 486460-32-6), SAXAGLIPTIN(CAS-No. 361442-04-8), VILDAGLIPTIN(CAS-No. 274901-16-5), DENAGLIPTIN(CAS-No. 483369-58-0), P32/98 (CAS-No. 251572-70-0) and NVP-DPP-728 (CAS-No. 247016-69-9).

Specific examples of PDE5 inhibitors include, but are not limited to SILDENAFIL (CAS-No. 139755-83-2), VARDENAFIL (CAS-No. 224785-90-4) and TADALAFIL (CAS-No. 171596-29-5). Examples of PDE1, PDE9, PDE10 or PDE11 inhibitors which may be usefully employed according to the present invention can be found, for example, in US20020160939, WO2003037432, US2004220186, WO2005/003129, WO2005012485, WO2005120514 and WO03077949.

A specific example of an amylin agonist includes, but is not limited to PRAMLINITIDE (CAS-No. 151126-32-8).

A specific example of a Coenzyme A inhibitor includes, but is not limited to ETOMOXIR(CAS-No. 082258-36-4).

Specific examples of anti-obesity drugs include, but are not limited to HMR-1426 (CAS-No. 262376-75-0), CETILISTAT (CAS-No. 282526-98-1) and SIBUTRAMINE (CAS-No. 106650-56-0).

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be determined approximately from height and weight of the patient. See, e.g., Scientific Tables, Geigy Pharmaceuticals, Ardsley, N.Y., 1970, 537.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for pentoxifylline.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

### Methods of Treatment

It has now been found that the M-2 metabolite of pentoxifylline is active in a TNF-α and an ROS assay (see Example 2). Accordingly, in one embodiment, the invention is a compound described herein for use in a medicament or for use as a therapeutic substance.

In one embodiment, the invention provides a method of inhibiting the activity of phosphodiesterase (PDE) in a cell, comprising contacting a cell with one or more compounds of Formula I.

In addition to its PDE inhibitory activity, pentoxifylline is known to suppress the production of a number of other biological agents such as interleukin-6 (IL-6), TNF-α, and various growth factors such as CTGF (connective tissue growth factor). Accordingly, in another embodiment, the invention provides a method of suppressing the production of IL-6, TNF-α, and various growth factors, such as CTGF (connective tissue growth factor), in a cell, comprising contacting a cell with one or more compounds of Formula I.

In addition, the invention provides a method of suppressing the production of MCP-1 and IFN-gamma in a cell, comprising contacting a cell with one or more compounds of Formula I.

According to another embodiment, the invention provides a method of treating a disease in a patient in need thereof that is beneficially treated by pentoxifylline comprising the step of administering to said patient an effective amount of a compound of Formula I or a pharmaceutical composition comprising a compound of Formula I and a pharmaceutically acceptable carrier.

Alternatively, the invention provides a method of treating a disease in a patient in need thereof that is beneficially treated by pentoxifylline comprising the step of administering per day to said patient an effective amount of a compound of Formula I or a pharmaceutical composition comprising a compound of Formula I and a pharmaceutically acceptable carrier.

Such diseases are well known in the art and are disclosed in, but not limited to the following patents and published applications: WO 1988004928, EP 0493682, U.S. Pat. No. 5,112,827, EP 0484785, WO 1997019686, WO 2003013568, WO 2001032156, WO 1992007566, WO 1998055110, WO 2005023193, U.S. Pat. No. 4,975,432, WO 1993018770, EP 0490181, and WO 1996005836. Such diseases include, but are not limited to, peripheral obstructive vascular disease; glomerulonephritis; nephrotic syndrome; nonalcoholic steatohepatitis; Leishmaniasis; cirrhosis; liver failure; Duchenne's muscular dystrophy; late radiation induced injuries; radiation induced lymphedema; radiation-associated necrosis; alcoholic hepatitis; radiation-associated fibrosis; necrotizing enterocolitis in premature neonates; diabetic nephropathy, hypertension-induced renal failure, and other chronic kidney disease; Focal Segmental Glomerulosclerosis; pulmonary sarcoidosis; recurrent aphthous stomatitis; chronic breast pain in breast cancer patients; brain and central nervous system tumors; malnutrition-inflammation-cachexia syndrome; interleukin-1 mediated disease; graft versus host reaction and other allograft reactions; diet-induced fatty liver conditions, atheromatous lesions, fatty liver degeneration and other diet-induced high fat or alcohol-induced tissue-degenerative conditions; human immunodeficiency virus type 1 (HIV-1) and other human retroviral infections; multiple sclerosis; cancer; fibroproliferative diseases; fungal infection; drug-induced nephrotoxicity; collagenous colitis and other diseases and/or conditions characterized by elevated levels of platelet derived growth factor (PDGF) or other inflammatory cytokines; endometriosis; optic neuropathy and CNS impairments associated with acquired immunodeficiency syndrome (AIDS), immune disorder diseases, or multiple sclerosis; autoimmune disease; upper respiratory viral infection; depression; urinary incontinence; irritable bowel syndrome; septic shock; Alzheimers Dementia; neuropathic pain; dysuria; retinal or optic nerve damage; peptic ulcer; insulin-dependent diabetes; non-insulin-dependent diabetes; diabetic nephropathy; metabolic syndrome; obesity; insulin resistance; dyslipidemia; pathological glucose tolerance; hypertension; hyperlipidemia; hyperuricemia; gout; hypercoagulability; acute alcoholic hepatitis; olfaction disorders; patent ductus arteriosus; and inflammation or injury associated with neutrophil chemotaxis and/or degranulation.

The compounds of Formula I can also be used to control intraocular pressure or to stabilize auto-regulation of cerebral blood flow in subjects who require such control as determined by medical examination.

In one particular embodiment, the method of this invention is used to treat a disease or condition in a patient in need thereof selected from intermittent claudication on the basis of chronic occlusive arterial disease of the limbs and other peripheral obstructive vascular diseases; glomerulonephritis; Focal Segmental Glomerulosclerosis; nephrotic syndrome; nonalcoholic steatohepatitis; Leishmaniasis; cirrhosis; liver failure; Duchenne's muscular dystrophy; late radiation induced injuries; radiation induced lymphedema; alcoholic hepatitis; radiation-induced fibrosis; necrotizing enterocolitis in premature neonates; diabetic nephropathy, hypertension-induced renal failure and other chronic kidney diseases; pulmonary sarcoidosis; recurrent aphthous stomatitis; chronic breast pain in breast cancer patients; brain and central nervous system tumors; obesity; acute alcoholic hepatitis; olfaction disorders; endometriosis-associated infertility; malnutrition-inflammation-cachexia syndrome; and patent ductus arteriosus.

In one embodiment, the method of this invention is used to treat diabetic nephropathy, hypertensive nephropathy or intermittent claudication on the basis of chronic occlusive arterial disease of the limbs. In another particular embodiment, the method of this invention is used to treat a disease or condition in a patient in need thereof selected from intermittent claudication on the basis of chronic occlusive arterial disease of the limbs.

In one embodiment, the method of this invention is used to treat chronic kidney disease. The chronic kidney disease may be selected from glomerulonephritis, focal segmental glomerulosclerosis, nephrotic syndrome, reflux uropathy, or polycystic kidney disease.

In one embodiment, the method of this invention is used to treat chronic disease of the liver. The chronic disease of the liver may be selected from nonalcoholic steatohepatitis, fatty liver degeneration or other diet-induced high fat or alcohol-induced tissue-degenerative conditions, cirrhosis, liver failure, or alcoholic hepatitis.

In one embodiment, the method of this invention is used to a diabetes-related disease or condition. This disease may be selected from insulin resistance, retinopathy, diabetic ulcers, radiation-associated necrosis, acute kidney failure or drug-induced nephrotoxicity.

In one embodiment, the method of this invention is used to treat a patient suffering from cystic fibrosis, including those patients suffering from chronic Pseudomonas bronchitis.

In one embodiment, the method of this invention is used to aid in wound healing. Examples of types of wounds that may be treated include venous ulcers, diabetic ulcers and pressure ulcers.

In another particular embodiment, the method of this invention is used to treat a disease or condition in a patient in need thereof selected from insulin dependent diabetes; non-insulin dependent diabetes; metabolic syndrome; obesity; insulin resistance; dyslipidemia; pathological glucose tolerance; hypertension; hyperlipidemia; hyperuricemia; gout; and hypercoagulability.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In one embodiment of the methods disclosed herein, the compound of Formula I is a compound disclosed in Table 1 herein.

In another embodiment, any of the above methods of treatment comprises the further step of co-administering to the patient an effective amount of one or more second therapeutic agents. The choice of second therapeutic agent may be made from any second therapeutic agent known to be useful for co-administration with pentoxifylline. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

### The Second

In particular, the combination therapies of this invention include co-administering an effective amount of a compound of Formula I and an effective amount of a second therapeutic agent for treatment of the following conditions (with the particular second therapeutic agent indicated in parentheses following the indication): late radiation induced injuries (α-tocopherol), radiation-induced fibrosis (α-tocopherol), radiation induced lymphedema (α-tocopherol), chronic breast pain in breast cancer patients (α-tocopherol), type 2 diabetic nephropathy (captopril), malnutrition-inflammation-cachexia syndrome (oral nutritional supplement, such as Nepro; and oral anti-inflammatory module, such as Oxepa); and brain and central nervous system tumors (radiation therapy and hydroxyurea).

The combination therapies of this invention also include co-administering an effective amount of a compound of Formula I and an effective amount of a second therapeutic agent for treatment of insulin dependent diabetes; non-insulin dependent diabetes; metabolic syndrome; obesity; insulin resistance; dyslipidemia; pathological glucose tolerance; hypertension; hyperlipidemia; hyperuricemia; gout; and hypercoagulability.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Example 1

### Synthesis of 1-(5,6-Dihydroxyhexyl)-3,7-dimethyl-xanthine (Formula Ia, Compound 308) (which is the M-2 metabolite of pentoxifylline)

Compound 308, which is the compound of formula I in which each Z is hydrogen, R³ is hydrogen, R¹ and R² are each CH₃, and any atom not designated as deuterium is at its natural isotopic abundance, was prepared as outlined in Scheme 2 and as described below. The preparation of compounds of formula I in which one or more Z is deuterium, such as the compound of formula I in which each Z is deuterium, R³ is hydrogen, and R¹ and R² are each CH₃, may be readily envisioned by the skilled artisan.

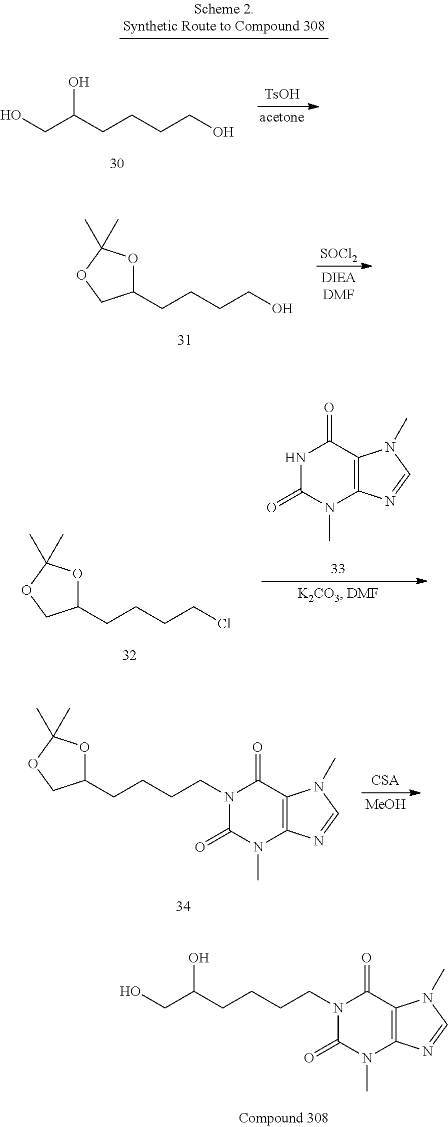

### Step 1. 4-(2,2-Dimethyl-1,3-dioxolan-4-yl)butan-1-ol (31)

Hexane-1,2,6-triol (30) (1.5 g, 10 mmol, commercially available) was dissolved in acetone (20 mL) and p-toluenesulfonic acid (100 mg) was added. The solution was stirred at room temperature for 6 hours. After concentration under vacuum, the crude product was purified by column chromatography on silica gel, eluting with 1:1 EtOAc/heptanes, to give 0.9 g (52%) of 31 as a colorless oil.

### Step 2. 4-(4-chlorobutyl)-2,2-dimethyl-1,3-dioxolane (32)

Intermediate 31 (0.8 g, 4.6 mmol) was dissolved in dry DMF (6 mL) and DIPEA (1 mL) was added. The solution was cooled in an ice-water bath and thionyl chloride (0.4 mL) was added. The reaction mixture was stirred at ice-water bath temperature for 1.5 hours. CH₂Cl₂ (60 mL) was added to the solution followed by saturated NaHCO₃ (15 mL). The organic phase was separated and the aqueous phase was extracted with CH₂Cl₂ (2×20 mL). The combined organic phases were washed with water (2×20 mL) and brine (2×20 mL). After drying (Na₂SO₄), the solvent was removed under reduced pressure. The crude product was purified by column chromatography on silica gel, eluting with 1:4 EtOAc/heptanes, to give 0.68 g (77%) of 32.

### Step 3. 1-(4-(2,2-Dimethyl-1,3-dioxolan-4-yl)butyl)-3,7-dimethyl-1H-purine-2,6(3H,7H)-dione (34)

3,7-Dimethyl-1H-purine-2,6(3H,7H)-dione (33) (540 mg, 3 mmol), intermediate 32 (0.66 g, 3.4 mmol) and powdered K₂CO₃ (0.82 g, 6 mmol) were mixed in DMF (6 mL). The mixture was heated at 110° C. for 2 hours. Stirring became difficult due to the formation of a sticky solid. More DMF (10 mL) was added to facilitate stirring. The mixture was kept at 85° C. overnight. After cooling, the mixture was passed through a pad of Celite and the pad was washed with MeOH (100 mL). The filtrate was concentrated to give a sticky yellow oil. The oil was dissolved in CH₂Cl₂ (200 mL) and the solution was washed with water (2×20 mL) and brine. After drying (Na₂SO₄), the solvent was removed under reduced pressure. The crude product was purified by column chromatography on silica gel, eluting with ethyl acetate, to give 590 mg (66%) of 34.

### Step 4. 1-(5,6-Dihydroxyhexyl)-3,7-dimethyl-1H-purine-2,6(3H,7H)-dione (Compound 308)

Intermediate 34 (500 mg) was dissolved in MeOH (10 mL), camphorsulfonic acid (50 mg) was added and the solution was stirred overnight. The solution was concentrated to dryness. Fresh methanol (10 mL) was added to the residue and the mixture was stirred 1 hour before being concentrated to dryness. The crude product was purified by column chromatography on silica gel, eluting with 4-8% MeOH/CH₂Cl₂, to afford a colorless sticky oil. The oil was crystallized from CH₂Cl₂ to give 300 mg (68%) of Compound 308 as a white solid.

¹H-NMR (300 MHz, CDCl₃): δ 1.38-1.58 (m, 5H), 1.68-1.74 (m, 4H), 2.23 (s, J=5.1, 1H), 2.44 (d, J=3.8, 1H), 3.42-3.50 (m, 1H), 3.58 (s, 3H), 3.63 (dt, J=3.3, 7.8, 1H), 3.67-3.73 (m, 1H), 3.99 (s, 3H), 4.04 (t, J=7.3, 2H), 7.52 (s, 1H). ¹³C-NMR (75 MHz, CDCl₃): −0.001, 22.52, 27.76, 29.75, 32.49, 33.64, 40.84, 66.63, 71.99, 141.52. HPLC (method: Waters Atlantis T3 2.1×50 mm 3 μm C18-RP column−gradient method 5−95% ACN+0.1% formic acid in 14 min (1.0 mL/min) with 4 min hold at 95% ACN+0.1% formic acid; Wavelength: 254 nm): retention time: 2.55 min; >99% purity. MS (M+H): 297.3.

### Example 2

### Biological Evaluation

The biological activity of compounds 308, 401 and 400 (pentoxifylline) was determined in a number of assays as disclosed below. Compound 308 is a metabolite of Compound 400, and is also referred to as “M-2” in this application

### TNF-α and ROS Biological Assays

TNF-α inhibition and ROS inhibition activities are both relevant to the pathology of kidney disease (see Costantini, Todd W. et al., Immunopharmacology and Immunotoxicology, 2009, 1-10; Kitada, Munehiro et al., Diabetes 2003, 52:2603; and Navarro-Gonzalez, J F et al., Nat Rev Nephrol 2011, 7:327-340; all of which are enclosed herewith as Exhibits F-H).

### Assay Protocols:

1) TNF-α Inhibition Assay Protocol

Whole blood (sodium heparin vacutainer) was obtained from two normal, healthy donors from Research Blood Components, Boston, Mass. For each donor sample, two duplicate assays were performed according to the following procedure. Blood was diluted 1:1 with Opti-MEM® Reduced Serum Medium (Invitrogen) and 100 μl of diluted blood was added to wells of a 96-well plate. The test compounds were serially diluted in Opti-MEM® to create a dose-response. The resulting diluted solutions (50 μl) were then added to the wells containing diluted blood and the mixture was incubated for 15 minutes at 37° C., 5% CO₂. Lipopolysaccharide (LPS) strain 113:H10 (obtained from Associates of Cape Cod #E0005) was prepared at a 4× concentration and 50 μl of 4 ng/ml solution was added to the blood to achieve a final concentration of 1 ng/ml. Control wells contained diluted blood and 100 μl Opti-MEM® (negative control) or 50 μl Opti-MEM® plus 50 μl LPS (positive control). Plates were then incubated for 24 h at 37° C., 5% CO₂. After incubation, diluted plasma was harvested by centrifugation at 3000 RPM for 2 minutes to pellet the blood cells. Supernatant (diluted plasma) was then transferred into a clean 96-well plate. The diluted plasma was further diluted 1:10 with ELISA reagent diluents and the TNF-α level for each compound was measured following manufacturer's instructions for the DuoSet® ELISAs (R & D Systems). The IC₅₀ value for the compound was calculated using commercially available statistics software and the average IC₅₀ values from the two donors was calculated. The potency of the compound relative to the potency of compound 401 was obtained by dividing the IC₅₀ value of compound 401 by the IC₅₀ value of the compound.

2) ROS Inhibition Assay Protocol

Whole blood (sodium heparin vacutainer) was obtained from two normal, healthy donors from Research Blood Components, Boston, Mass. For each donor sample, two duplicate assays were performed according to the following procedure. Whole blood (100 μl) was added to wells of a 96 deep-well plate. Compounds were serially diluted in Hanks Balanced Salt Solution (HBSS) to achieve a desired dose-response. The resulting diluted solutions (100 μl) were added to the whole blood samples, mixed gently, and incubated for 30 minutes at 37° C., 5% CO₂. The blood was then stimulated by addition of Formyl-Methionyl-Leucyl-Phenylalanine (fMLP) (1 μM final concentration) or phorbol myristate acetate (PMA) (10 ng/ml final concentration) and was further incubated for 20 minutes at 37° C., 5% CO₂. Dihydrorhodamine 123 (DHR-123) reagent (Invitrogen), a cell-permeable probe that becomes highly fluorescent when oxidized, was then added to the blood at a final concentration of 0.5 μM and incubated for another 20 minutes at 37° C., 5% CO₂. The red blood cells were then lysed in 1 ml ammonium chloride-potassium (ACK) lysis buffer (Invitrogen) for 10 minutes at room temperature. The remaining leukocytes (including the neutrophils) were washed once in HBSS and resuspended in HBSS/0.5% paraformaldehyde. To quantify intracellular ROS production, the fluorescence of the oxidized form of DHR-123, which is a fluorescent compound, was measured. The level of oxidized DHR-123 in neutrophils was assessed for each sample by measuring cell fluorescence on a flow cytometer. The IC₅₀ value was calculated using commercially available statistics software and the average IC₅₀ values from the two donors was calculated. The potency of the compound relative to the potency of compound 401 was obtained by dividing the IC₅₀ value of compound 401 by the IC₅₀ value of the compound.

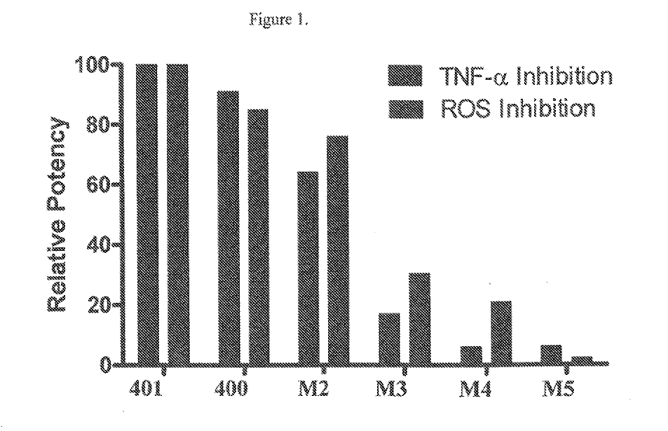

The results of the above TNF-α and ROS assays for compound 308 (M-2), compound 400 and compound 401 are shown in FIG. 1. As shown in the figure, the three compounds had comparable activity in both assays. The activity in the two assays is also shown for other metabolites of compound 400, indicated as M-3, M-4 and M-5 in FIG. 1. In FIG. 1, for each compound, the relative potency in the TNF-α inhibition assay is shown as the left-hand-side bar, while the relative potency in the ROS inhibition assay is shown as the right-hand-side bar. Metabolites M-3, M-4 and M-5 showed significantly lower activity in both assays relative to compounds 308, 400 and 401. The structures of M-3, M-4 and M-5 are shown below:

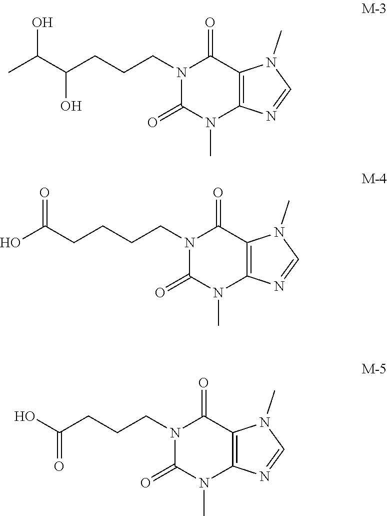

Because deuteration is not expected to affect pharmacological activity, the same relative activities are believed to be observable regardless of whether a deuterated or non-deuterated form of each species is tested.

### MCP-1 Assay

MCP-1 is a known pro-inflammatory cytokine that plays important roles in various diseases. Table 2 shows the MCP-1 IC₅₀ values for Compounds 308, 400 and 401 for the two blood donors that are referred to in Table 2.

| | 400 | 401 | 308 |
|---|---|---|---|
| MCP-1 | IC50 μM | IC50 μM | IC50 μM |
| Donor 1 | 80 | 120 | 194 |
| Donor 2 | 80 | 69 | 125 |

As shown in Table 2, the IC₅₀ values for Compound 308 were found to be slightly higher than for Compounds 400 and 401. The MCP-1 IC₅₀ trend is similar to that observed for TNF-α.

### IFN-Gamma Assay

Table 3 shows the IFN-gamma IC₅₀ values for Compounds 308, 400 and 401 for two different blood donors.

| | 400 | 401 | 308 |
|---|---|---|---|
| IFN-gamma | IC50 μM | IC50 μM | IC50 μM |
| Donor I | 78 | 70 | 72 |
| Donor II | 63 | 31 | 119 |

As shown in Table 3, the IC₅₀ values for Compound 308 were found to be slightly higher than for Compounds 400 and 401 for one donor, and very similar for a second donor. The IFN-gamma IC₅₀ trend is similar to that observed for TNF-α and MCP-1.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

We claim:

1. A pharmaceutical composition comprising a compound of Formula I: 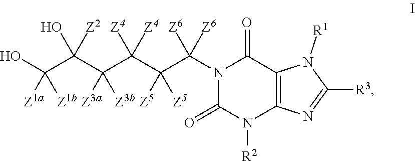
or a pharmaceutically acceptable salt thereof, wherein:
each of R¹ and R² is independently selected from —CH₃ and —CD₃;
R³ is hydrogen or deuterium;
each of Z¹, Z² and Z³ is independently selected from hydrogen and deuterium;
each Z⁴ is hydrogen or deuterium;
each Z⁵ is hydrogen or deuterium; and
each Z⁶ is hydrogen or deuterium.

2. The composition of claim 1 wherein at least one of R¹ and R² is —CD₃ or at least one of R³, Z¹, Z², Z³, Z⁴, Z⁵ and Z⁶ is deuterium.

3. The composition of claim 1 wherein each Z⁴, Z⁵ and Z⁶ is hydrogen.

4. The composition of claim 1 wherein each Z², Z⁴, Z⁵ and Z⁶ is hydrogen.

5. The composition of claim 1 wherein each Z¹ and Z³ is deuterium and each Z², Z⁴, Z⁵ and Z⁶ is hydrogen.

6. The composition of claim 1 wherein each of R¹ and R² is —CH₃ and R³ is hydrogen.

7. The composition of claim 1, wherein the compound is the enantiomer having the (S) configuration at the carbon bearing the Z² substituent.

8. The composition of claim 1, wherein the compound is 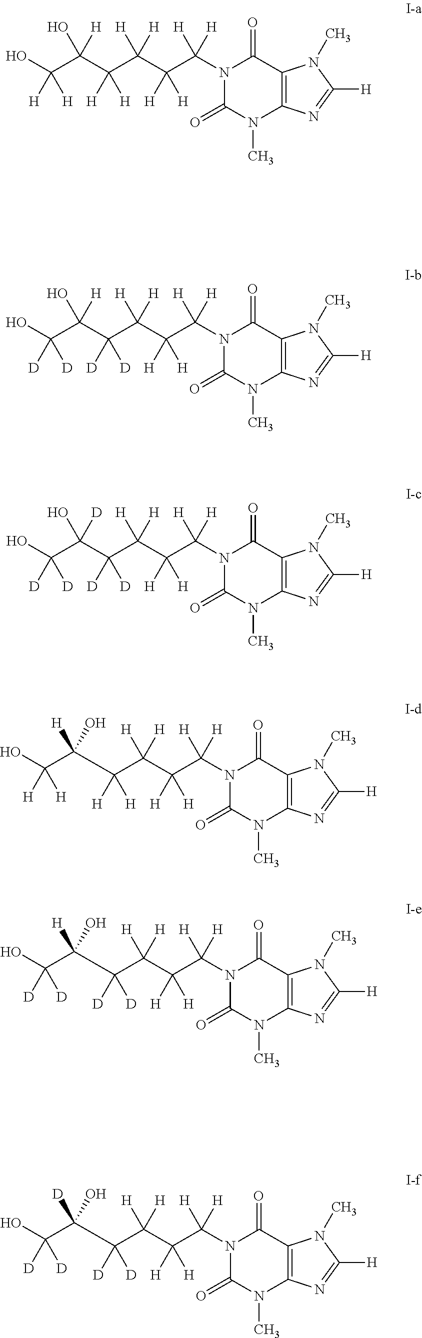 [SMILES: Cn1cnc2c1c(=O)n(CCCCC(O)CO)c(=O)n2C ; [2H]C([2H])(O)C(O)C([2H])([2H])CCCn1c(=O)c2c(ncn2C)n(C)c1=O ; [2H]C([2H])(O)C([2H])(O)C([2H])([2H])CCCn1c(=O)c2c(ncn2C)n(C)c1=O ; Cn1cnc2c1c(=O)n(CCCC[C@@H](O)CO)c(=O)n2C ; [2H]C([2H])(O)[C@H](O)C([2H])([2H])CCCn1c(=O)c2c(ncn2C)n(C)c1=O ; [2H]C([2H])(O)[C@]([2H])(O)C([2H])([2H])CCCn1c(=O)c2c(ncn2C)n(C)c1=O] 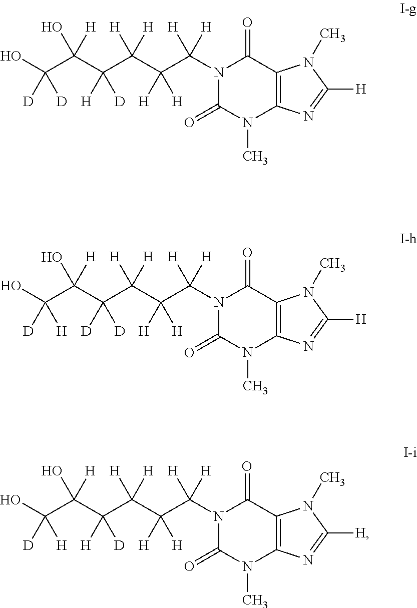
or a pharmaceutically acceptable salt thereof.

9. The composition of claim 1, wherein any atom not designated as deuterium in any of the embodiments set forth above is present at its natural isotopic abundance.

10. A compound of Formula I: 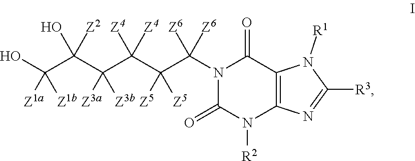
or a pharmaceutically acceptable salt thereof, wherein:
each of R¹ and R² is independently selected from —CH₃ and —CD₃;
R³ is hydrogen or deuterium;
each of Z¹, Z² and Z³ is independently selected from hydrogen and deuterium;
each Z⁴ is hydrogen or deuterium;
each Z⁵ is hydrogen or deuterium; and
each Z⁶ is hydrogen or deuterium; and
wherein the compound is at least 90% pure by weight.

11. The composition of claim 2, wherein the compound is at least 90% pure by weight.

12. A method of treating a disease or condition in a patient in need thereof, comprising administering to the patient an effective amount of a composition of claim 1, wherein the disease is selected from diabetic nephropathy, hypertensive nephropathy or intermittent claudication on the basis of chronic occlusive arterial disease of the limbs.

13. A method of treating chronic kidney disease in a patient in need thereof, comprising administering to the patient an effective amount of a composition of claim 1.

14. The method of claim 13 wherein the chronic kidney disease is glomerulonephritis, focal segmental glomerulosclerosis, nephrotic syndrome, reflux uropathy, or polycystic kidney disease.

15. A method of treating chronic disease of the liver in a patient in need thereof, comprising administering to the patient an effective amount of a composition of claim 1.

16. The method of claim 15 wherein the chronic disease of the liver is nonalcoholic steatohepatitis, fatty liver degeneration or other diet-induced high fat or alcohol-induced tissue-degenerative conditions, cirrhosis, liver failure, or alcoholic hepatitis.

17. A method of treating a diabetes-related disease or condition in a patient in need thereof, comprising administering to the patient an effective amount of a composition of claim 1, wherein the disease or condition is selected from insulin resistance, retinopathy, diabetic ulcers, radiation-associated necrosis, acute kidney failure or drug-induced nephrotoxicity.

18. A method of treating intermittent claudication in a patient in need thereof, comprising administering to the patient an effective amount of a composition of claim 1.

19. A method of treating a disease or condition in a patient in need thereof, wherein the disease or condition is selected from insulin dependent diabetes; non-insulin dependent diabetes; metabolic syndrome; obesity; insulin resistance; dyslipidemia; pathological glucose tolerance; hypertension; hyperlipidemia; hyperuricemia; gout; and hypercoagulability, comprising administering to the patient an effective amount of a composition of claim 1.

20. The compound of claim 10, wherein the compound is selected from the group consisting of 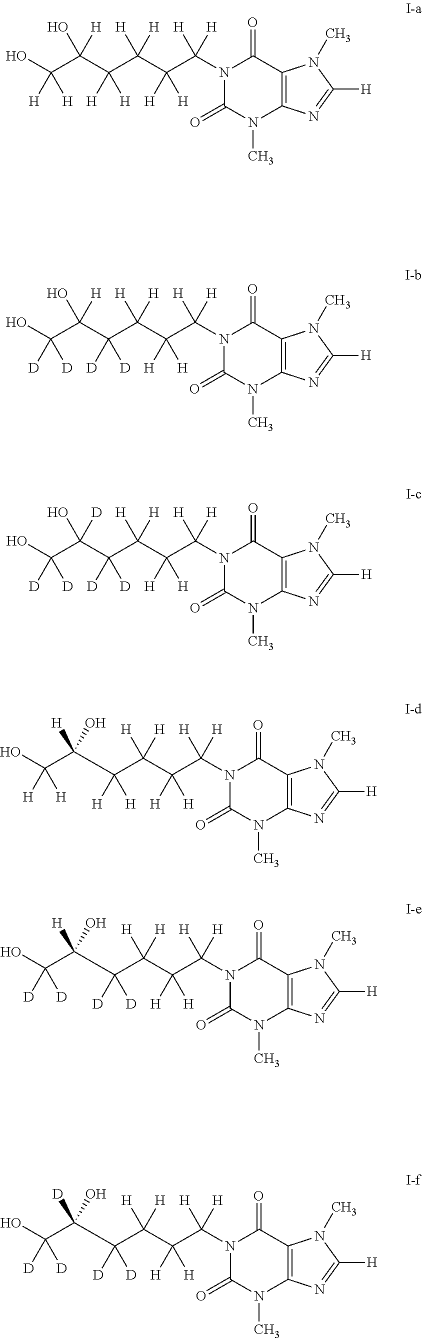 [SMILES: Cn1cnc2c1c(=O)n(CCCCC(O)CO)c(=O)n2C ; [2H]C([2H])(O)C(O)C([2H])([2H])CCCn1c(=O)c2c(ncn2C)n(C)c1=O ; [2H]C([2H])(O)C([2H])(O)C([2H])([2H])CCCn1c(=O)c2c(ncn2C)n(C)c1=O ; Cn1cnc2c1c(=O)n(CCCC[C@@H](O)CO)c(=O)n2C ; [2H]C([2H])(O)[C@H](O)C([2H])([2H])CCCn1c(=O)c2c(ncn2C)n(C)c1=O ; [2H]C([2H])(O)[C@]([2H])(O)C([2H])([2H])CCCn1c(=O)c2c(ncn2C)n(C)c1=O] 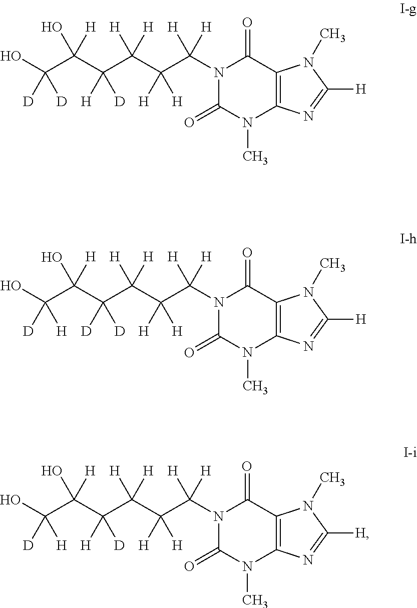
I-i, or a pharmaceutically acceptable salt thereof.
